(19) United States
(12) Patent Application Publication
(10) Pub. No.: US 2022/0015437 A1
(43) Pub. Date: Jan. 20, 2022

(54) E-CIGARETTE

(71) Applicant: SMOKELESS.WORLD GMBH, Grünwald (DE)

(72) Inventors: Daniel KILGER, München (DE); Michael SEDLMEIER, München (DE); Patrick MÜLLER, Heiningen (DE)

(21) Appl. No.: 17/295,502

(22) PCT Filed: Nov. 18, 2019
(86) PCT No.: PCT/EP2019/081654

§ 371 Date: May 20, 2021

(30) Foreign Application Priority Data
Nov. 22, 2018 (EP) 18207886.5

(51) Int. Cl. A24F 40/50; G16H 20/13; A24F 40/10; A24F 40/30; A24F 40/42; A24F 40/65

(52) U.S. Cl. CPC A24F 40/50; G16H 20/13; A24F 40/10; A61M 15/06; A24F 40/42; A24F 40/65; A24F 40/30

(57) ABSTRACT

The present invention is directed to a system for dosing an inhaling composition. It is preferably integrated into an e-cigarette. The system comprises more than one component for the inhaling composition. A first container (30) can be provided for a first component for the composition. A second container (31) can be further provided for a second component for the composition. Moreover, a dosing arrangement (10, 11, 40) can be arranged that is configured to automatically reduce the dose of at least the second component over time.

### FIELD

The present invention relates to an inhaler, such as a portable inhaler or more specifically to an e-cigarette, for automatically dosing at least one component, such as nicotine or cannabis, in an inhaling composition with more than one component.

### BACKGROUND

According to Wikipedia of October 2018, an electronic cigarette or e-cigarette is a handheld electronic device that simulates the feeling of smoking. It works by heating a liquid to generate an aerosol, commonly called a “vapor”, that the user inhales. Using e-cigarettes is commonly referred to as vaping. The liquid in the e-cigarette, called e-liquid, or e-juice, is usually made of nicotine, propylene glycol, glycerine, and flavorings. Not all e-liquids contain nicotine.

E-cigarettes can create an aerosol, commonly called vapor. Its exact composition varies. The majority of toxic chemicals found in tobacco smoke are absent in e-cigarette aerosol. Those present are mostly below 1% of the corresponding levels in tobacco smoke. The aerosol can contain toxicants and traces of heavy metals at levels permissible in inhalation medicines, and potentially harmful chemicals not found in tobacco smoke at concentrations permissible by workplace safety standards.

The modern e-cigarette was invented in 2003 by Chinese pharmacist Mr. Hon Lik, and as of 2018 most e-cigarettes are made in China. Since they were first sold in 2004 their global use has risen exponentially. In the United States and the United Kingdom their use is widespread. Reasons for using e-cigarettes involve trying to quit smoking, reduce risk, or save money, though some use them recreationally. As of 2014, the majority of users still smoke tobacco. There are concerns that dual use of tobacco products and e-cigarettes may “delay or deter quitting”. About 60% of UK users are smokers and roughly 40% are ex-smokers. In the UK use among never-smokers was negligible.

Electronic cigarettes are also known as e-cigarettes, e-cigs, EC, electronic nicotine delivery systems (ENDS) or electronic non-nicotine delivery systems (ENNDS), electronic smoking devices (ESDs), personal vaporizers, or PVs. They are handheld devices, often made to look like conventional cigarettes, and used in a similar way.

There are three main types of e-cigarettes: cigalikes, looking like cigarettes; eGos, bigger than cigalikes with refillable liquid tanks; and mods, assembled from basic parts or by altering existing products. As the e-cigarette industry continues to evolve, new products are quickly developed and brought to market. First generation e-cigarettes tend to look like tobacco cigarettes and so are called “cigalikes”. Most cigalikes look like cigarettes but there is some variation in size. A traditional cigarette is smooth and light while a cigalike is rigid and slightly heavier. Second generation devices are larger overall and look less like tobacco cigarettes. Third generation devices include mechanical mods and variable voltage devices. The fourth generation includes Sub ohm tanks and temperature control devices. The power source is the biggest component of an e-cigarette, which is frequently a rechargeable lithium-ion battery.

The main components of an e-cigarette are a mouthpiece, a cartridge (tank), a heating element/atomizer, a microprocessor, a battery, and possibly an LED light on the end. The only exception to this are mechanical e-cigarettes (mods) which contain no electronics; the circuit is closed by a mechanical action switch. An atomizer comprises a small heating element, or coil, that vaporizes e-liquid and wicking material that draws liquid onto the coil. When the user pushes a button, or (in some variations) activates a pressure sensor by inhaling, the heating element atomizes the liquid solution. The e-liquid reaches a temperature of roughly 100-250° C. within a chamber to create an aerosolized vapor, which the user then inhales, rather than cigarette smoke. The aerosol provides a flavor and feel similar to tobacco smoking.

E-liquid or juice are names for the flavored solution that goes inside the e-cigarette. An aerosol, or vapor, is produced by heating the e-liquid. Irish public health discussions refer to NMNDS (“non-medicinal nicotine delivery systems”). When the FDA commissioned their 2018 report on ENDS which they label as a Tobacco Product, the authors chose to use the term e-cigarettes for some use e-juice without nicotine.

E-liquid is the mixture used in vapor products such as e-cigarettes and generally consists of propylene glycol, glycerin, water, nicotine, and flavorings. While the ingredients vary the liquid typically contains 95% propylene glycol and glycerin. There are many e-liquids manufacturers in the US and worldwide, and upwards of 8,000 flavors. Industry standards have been created and published by the American E-liquid Manufacturing Standards Association (AEMSA).

Between their introduction to the market in 2004 and approximately 2015, global usage of e-cigarettes rose exponentially. By 2013, there were several million users globally. Awareness and use of e-cigarettes greatly increased in a relatively short period of time. Growth rates in the US and UK slowed in 2015, although use is still increasing.

Most users have a history of smoking regular cigarettes. At least 52% of smokers or ex-smokers have vaped. Of smokers who have, one British study reported that less than 15% became everyday e-cigarette users. One United States survey of e-cigarette users conducted from 2011-2012 found that only 1% of respondents used liquid without nicotine.

E-cigarettes may be used with other substances and cartridges can potentially be filled with e-liquid containing substances other than nicotine, thus serving as a new way to deliver other psychoactive drugs, for example cannabis.

Cannabis, also known as marijuana among other names, is a psychoactive drug from the Cannabis plant or synthesized used for medical or recreational purposes. The main psychoactive part of cannabis is tetrahydrocannabinol, one of 483 known compounds in the plant, including at least 65 other cannabinoids. Cannabis can be used by smoking, vaporizing, within food, or as an extract. The term cannabis is intended to cover THC, CBD or cannabinoids, terpenes etc.

The emergence of e-cigarettes has given cannabis smokers a new method of inhaling cannabinoids. E-cigarettes, also known as vape pens, cartridges and pens, differ from traditional marijuana cigarettes in several respects. It is assumed that vaporizing cannabinoids at lower temperatures is safer because it produces smaller amounts of toxic substances than the hot combustion of a marijuana cigarette. Recreational cannabis users can discreetly “vape” deodorized cannabis extracts with minimal annoyance to the people around them and less chance of detection, known as “stealth vaping”. While cannabis is not readily soluble in the liquid used for e-cigs, recipes containing synthetic cannabinoids which are soluble may be found.

EP 2 608 686 B1 is directed to an inhalation device comprising two cassettes with two dispensing devices with each being associated to one ingredient. The inhalation device can estimate the number of cigarette equivalents remaining based on an estimation of the remaining ingredient and display this by means of a display.

Furthermore, the U.S. Pat. No. 7,028,693 B2 provides a procedure for the weaning of smoking articles, in which a cigarette dispenser is provided to control the dispensing of cigarettes out of a cigarette pack.

WO 2015 150 699 A1 describes a device for setting into a portable terminal device a quantity of an active substance inhaled by a user. For the mentioned adjustment of the amount of active substance the volume of steam inhaled by the user is measured.

### SUMMARY

It is the object of the present invention to provide an improved or alternative mobile inhaler, particularly an e-cigarette, and a respective method for an improved or alternative way of inhaling from a mobile inhaler, particularly from an e-cigarette.

It is another optional object to provide a mobile inhaler, particularly an e-cigarette, and a respective method of inhaling that allow a more flexible way of inhaling and a respective method of inhaling.

It is another optional object of the present invention to provide a mobile inhaler, particularly an e-cigarette, and a respective method of inhaling that allow to automatically and to flexibly mix ingredients in an inhaling composition and/or control one or more components from the composition.

It is another optional object of the present invention to provide a mobile inhaler, particularly an e-cigarette, and a respective method of inhaling that allow to automatically reduce harmful and/or addictive components from the inhaling composition over time and, optionally, to allow a user to stop use of addictive components, such as nicotine and/or cannabis.

It is another optional object to provide an assembly of an e-cigarette that can be controlled in an optimized manner and to inter alia make use of other components.

It is a still further optional object to provide an assembly of an e-cigarette, a handheld device enabling better handling by a user and further computing abilities and power.

Each of the above objects is attained with the subject matter of the present invention as recited in the claims, embodiments and/or description.

The present invention is directed to a system for dosing an inhaling composition. This system can also be called a dosing system. It is preferably integrated into an e-cigarette. Thus, the invention is preferably directed to an e-cigarette with the features mentioned below.

The system comprising can comprise more than one component for the inhaling composition. A first container can be provided fora first component for the composition. A second container can be further provided for a second component for the composition. One or more container can be also provided with further potential components for the inhaling composition.

Moreover, a dosing arrangement can be provided that is configured to automatically control, preferably reduce the dose of at least the second component over time.

In an independent aspect the invention can alternatively or additionally also comprise a dosing arrangement that is configured to dose at least the second component in time intervals of at most 200 ms.

The system is a mobile device, preferably a battery driven mobile device and more preferably an electronic cigarette or e-cigarette. The battery is preferably integrated but can be also a separate element.

The dosing arrangement can be configured to reduce a number of inhales comprising the second component. As mentioned, the second component is preferably nicotine. Additionally or alternatively it can also be cannabis or any other similar component with mental or physical impact to users.

The dosing arrangement can be configured to reduce at least one of an amount of the second component over a number of inhales and a number of doses of the second component during an inhale. These are different approaches that can be combined as well. One is to provide some inhales with the second component and some without the second component. The second approach is to basically provide all inhales with the second component wherein the amount of the second component can be reduced over time.

The dosing arrangement can comprises a first aerosol generator that is configured to vaporize the first component to a first aerosol, preferably so as to provide a first aerosol as a carrier component together with air. The air is usually delivered anyhow, preferably with the sucking action of a user or can be provided automatically. Into the air usually the first aerosol can be delivered so that there is no dry air and so that the user does not feel any difference in consistency of the vapor in case the second component is delivered or not.

A mouth piece and a canal can be provided that are configured to supply air to the mouth piece wherein at least one of the first and second aerosol generator(s) can be configured to deliver at least one of the first and second aerosols to the canal.

The dosing arrangement can also comprise a first aerosol generator that can be configured to vaporize the first and the second components to a first and a second aerosol. In this case there is one aerosol generator for vaporizing or aerosolizing multiple components.

The dosing arrangement can also comprise a second aerosol generator that is configured to vaporize the second component to a second aerosol. Further aerosol generators can be also applied for further components.

The dosing arrangement can be configured to keep the amount or aerosol constant or essentially constant during an inhale. Then, the dosing arrangement can be configured to accordingly increase the amount aerosol based on the first component when the amount of aerosol based on the second component is reduced (this includes no second component). This will avoid dry air delivered to the user.

The dosing arrangement can comprise a controller that is configured to control at least one of the first and second aerosol generators. This can be done according to any controlling pattern. Preferably a reduction model is used. This can be adjustable as will be addressed further below.

The controller can be configured to at least control one of the activations of the first and second aerosol generators and the amount of aerosol generated and delivered to the air.

At least one of the first and second the aerosol generators can be configured to vaporize or aerosolize at least one of the first and second aerosols by heating or ultrasonic action.

At least one of the first and the second aerosol generators can comprise a porous material that is configured to be heated and that is configured to allow passage of at least one of the first and second components when it is heated.

The heating can be done by at least a porous member releasing at least one of the first and the second component as an aerosol when being heated. That porous member can have a gradient of porosity throughout the member and can release any of the components as an aerosol when the porous member is heated.

The dosing component can be configured to dose at least the second component in time intervals of at most 100 ms, preferably at most 50 ms, more preferably at most 35 ms, preferably at most 25 ms, more preferably at most 20 ms, more preferably at most 15 ms, even more preferably at most 10 ms and most preferably at most 7.

The dosing component can be further configured to dose in time intervals of at least 1 ms, preferably at least 2 ms, preferably at least 3 ms, more preferably at least 4 ms, even more preferably at least 5 ms, even more preferably at least 6 ms.

Preferably the dosing component is configured to dose in time intervals of between 1 ms and 15 ms, preferably between 2 ms and 20 ms, more preferably between 3 ms and 15 ms and most preferably between 5 ms and 10 ms. Thus, the dosing can be rather short and can supply the same amounts in pre-defined time intervals but not the entire time. This allows a sophisticated but rather simple dosing.

The dosing component can be configured to heat at least one of the first component and the second component to a temperature of more than 100° C., preferably at least 150° C., more preferably between 150° C. and 300° C., even more preferably between 180° C. and 260° C. and most preferably between 200° C. and 240° C.

Moreover, a boosting member can be provided and configured to overrule the dosing component by a user when activated to change the amount of the second component, particularly to increase the amount of the second component instantly. The boosting member can comprise at least one of a knob and any associated software switch, such as one installed in a software application installed on an associated handheld device.

The dosing component can also comprise a reduction model that is configured to cooperate with the controller in the controlling of the delivery of at least one of the first and second components. This model can be self-learning as will be specified later.

The first and second containers can be arranged in series along with at least one canal for delivering air to the user or the mouthpiece. The respective first and second aerosol generators can then be arranged in series along and in connection with the canal. This can preferably prevent that any dry delivery of air without any aerosol takes place. The container are preferably arranged around the air canal and/or preferably the aerosol generators are arranged in the air canal, at least in part.

The system can further comprise at least one of a mouth piece that is configured to be taken into the mouth of a user and a battery that is configured to deliver energy to at least one of the dosing arrangements. Then all element for an e-cigarette are provided.

Moreover, the dosing component can comprise a data storage. The data storage that is then arranged in the e-cigarette is basically used for the storage of the using pattern of the user and a model or lookup-table for the control, preferably the reduction of at least the second component. Moreover, personal data can be contained therein and the storage can also comprise a storage in a member outside the e-cigarette, such as a user's handheld device and/or the cloud.

A user interface can also be provided with at least one of an activation switch, an LED, a display, a fingerprint sensor, a face recognition sensor, a lip recognition sensor. The latter shall ensure that only the registered user is entitled to make use of the system. This can be particularly useful in case any therapeutically active components are delivered.

The system can further comprise a data storage that can also—at least in part—be outsourced, such as in a handheld device or remote server.

According to the present invention also an assembly can be provided that is composed of multiple components preferably comprising an e-cigarette according to the invention as described and claimed. The assembly can comprise the system according to the described or claimed dosing system and can further comprise at least one of a handheld device and a remote server. The e-cigarette can be configured to communicate with at least one of the handheld device and the remote server. In case it communicates directly with the remote server or cloud an IoT-e-cigarette is also addressed by the present invention. This would even make the handheld device unnecessary.

The system can be configured to communicate data to at least one of the handheld device and the remote server.

The system can further be configured to communicate behavior data to at least one of the handheld device and the remote server.

The behavior data can comprise smoking behavior data. That is, the behavior can be data relating to a smoking behavior of the user. The smoking behavior data can comprise usage data of the dosing system as well as data relating to a usage of conventional cigarettes.

The system can be configured to communicate puff data to at least one of the handheld device and the remote server.

The puff data can be data relating to puffs, that is inhalations.

The puff data can comprise data specifying at least one of a duration of puffs, a number of puffs, timestamps of puffs, in indicator for a ratio and/or an amount of nicotine in a puff, information whether a puff comprises nicotine at all and a usage of the boosting member.

The system can be configured to communicate device settings data to at least one of the handheld device and the remote server.

The device settings data can comprise data relating to an operation mode of the device.

The device settings data can comprise data relating to at least one setting of the device, such as whether factory settings are activated, whether the device is performing an analysis of a smoking behavior of the user and/or whether the device is reducing an output of nicotine.

In other words, the device settings data can comprise data relating to a nicotine dosage, a data collection and/or a phase of an analysis/reduction cycle.

The system can be configured to communicate device information data to at least one of the handheld device and the remote server.

The device information data can comprise data relating to a charge level of the battery of the system. The device data can also relate to a connection of the system, such as a connection of the system to the handheld device.

The device information data can also comprise data relating to an amount of the first and/or the second component of the composition.

These data relating to the amount of the first and/or the second component can be measured data.

These data relating to the amount of the first and/or the second component can also be estimated data, such as data estimated based on an activation of the dosing component or an element thereof.

These data relating to the amount of the first and/or the second component can also be data from a counter, for example a counter for an amount of puffs or a time of activation of the device or the dosage component.

The device data can also comprise location data relating to a location of the device.

The remote server can be configured to communicate data to at least one of the handheld device and the system.

The remote server can further be configured to communicate reduction algorithm data to at least one of the handheld device and the system.

The reduction algorithm data can comprise a portion of data of the reduction model.

The remote server can further be configured to communicate treatment data to at least one of the handheld device and the system. The treatment data can relate to phases of an operation of the system.

The remote server can also be configured to communicate update data to at least one of the handheld device and the system.

The update data can for example comprise update data for a software configured to operate the system.

The update data can also comprise data for a software configured to connect the handheld device and the system.

The handheld device can be configured to communicate data to the remote server.

The handheld device can be configured to communicate said behavior data to the remote server.

The handheld device can be configured to communicate said puff data to the remote server.

The handheld device can be configured to communicate the device settings data to the remote server.

The handheld device can be configured to communicate said device information data to the remote server.

The handheld device can be configured to communicate user data to the remote server.

The user data can be personal data of the user.

The user data can be personalized data of the user.

The handheld device can be configured to communicate treatment data to the remote server.

The treatment data can be any data which was input to the handheld device regarding the system, its operation or corresponding user data. That can for example also be smoking behavior of a user in response to data outputted by the handheld device or data from other handheld devices, such as a number of followers in a software application of the handheld device configured to control the system.

The handheld device can be configured to communicate data to the system.

The handheld device can be configured to communicate at least one of user identification data and user verification data to the system.

The handheld device is configured to communicate user input data to the system.

The user input data may comprise data relating to the smoking behavior, user adjustments to the device or its operation, or other user input data such as a use of conventional cigarettes.

The handheld device can be configured to communicate time data to the system. That is, the handheld device can be configured to communicate a current time to the system.

The handheld device can anyhow be a smart handheld device, such as a smart phone or a tablet computer.

Moreover, a software application can be provided and installed on the handheld device that is configured to introduce personalized data of an individual user, such as at least one of age, gender, weight, location, working profiles, smoking habits, number of boost activations per time, and that is making this personalized data available for the training of the reduction model. Other data can be used as well.

The software application can be configured to make data communicated to the system available for the training of the reduction model.

The software application can be configured to make empirical data available for the training of the reduction model.

At least one of the dosing system and the handheld device can be configured to collect user behavior data and to communicate the data to the remote server.

At least one of the personalized data and the user behavior data can be taken as a basis for modifying the reduction model for all users and/or for an individual user.

The remote server can be configured to collect at least one of user behavior data and personalized data, to compute or modify at least one reduction model and to transfer the reduction model to the dosing system, particularly to the dosing component. Thus, a self-learning approach can be realized in order to optimize reduction progress while still making it comfortable for the user.

The server can be configured to communicate at least one reduction model to the system.

The system can comprise a software application configured to control the system according to the reduction model.

The server can be configured to communicate updates for at least one of the at least one reduction model to the system.

The server can be configured to adapt at least one of the at least one reduction model based on data received from at least one of the system, the handheld device, a plurality of the systems and a plurality of the handheld devices, each corresponding to at least one system.

The handheld device can be also configured to display data of a plurality of users in order to provide extra motivation and illustration to the user.

Alternatively or additionally, the system according to any of the preceding system embodiments can be controlled by a remote controller. The remote controller can be particularly configured to control the dosing arrangement, particularly the dosage of the second component to be delivered.

This can be particularly useful in case a doctor preferably or necessarily has to control the delivery of the second component. This is particularly the case in case a user or patient is supposed to reduce the consumption of psychoactive drugs, such as cannabis. It can also serve a proper dosage of cannabinoids for therapeutic use in order to avoid any overdose.

The remote controller, such as a computer, a tablet or a second handheld device in the possession of a doctor can be configured to connect directly with the system or indirectly with the system over at least one of the handheld device of the user and a remote server and the cloud. The communication can be unidirectionally so that the doctor is determining the doses and/or bidirectionally so that the system is reporting back to the doctor the consumption behavior of the user.

The remote controller can also be the handheld device. That is, the system can be configured to be controlled from the handheld device. In such a case, the handheld device can comprise a software application configured to control the system. The software application can further be configured to accept corresponding input data. The remote controller can be configured to control by at least one encrypted command. This can ensure that misuse is prevented. The command can be delivered to the system by at least one of the system and the handheld device that are configured to decrypt the command and to provide the command to the controller of the system for controlling the dosing arrangement.

The remote controller can also be the handheld device. Thus, the system can be controlled from the handheld device.

The remote controller and the system can be configured to communicate with each other hardwired or wireless. In the latter case the remote controller and the system can be configured to communicate at least over one of wifi, Bluetooth, nfc. Other communication protocols can be used as well.

The present invention also relates to a respective method that makes use of all features and aspects of the system and the assembly as described and claimed.

### EMBODIMENTS

Below is a list of system embodiments. Those will be indicated with a letter “S”. Whenever such embodiments are referred to, this will be done by referring to “S” embodiments.
S1. A system for dosing an inhaling composition, the system comprising:
   a. a first container (30) for a first component for the composition;
   b. a second container (31) for a second component for the composition; and
   c. a dosing arrangement (10, 11, 40) that is configured to automatically control the dose of at least the second component over time.
S2. A system for dosing an inhaling composition, the system comprising:
   a. a first container (30) for a first component of the composition;
   b. a second container (31) for a second component of the composition; and
   c. a dosing arrangement (10, 11, 40) that is configured to dose at least the second component in time intervals of at most 200 ms.
S3. The system combining the two preceding system embodiments.
S4. The system according to any of the preceding embodiments wherein the dosing arrangement (10, 11, 40) is configured to automatically reduce the dose of at least the second component over time.
S5. The system according to any of the preceding system embodiments wherein the system is a mobile device, preferably a battery driven mobile device and more preferably an electronic cigarette or e-cigarette.
S6. The system according to any of the preceding system embodiments wherein the dosing arrangement (10, 11, 40) is configured to reduce a number of inhales comprising the second component.
S7. The system according to any of the preceding embodiments wherein the dosing arrangement (10, 11, 40) is configured to reduce at least one of an amount of the second component over a number of inhales and a number of doses of the second component during an inhale.
S8. The system according to any of the preceding system embodiments wherein the dosing arrangement (10, 11, 40) comprises a first aerosol generator (10, 11) that is configured to vaporize the first component to a first aerosol, preferably so as to provide a first aerosol as a carrier component together with air.
S9. The system according to any of the preceding embodiments further comprising a mouth piece (1) and a canal (3) that is configured to supply air to the mouth piece (1) wherein at least one of the first and second aerosol generator(s) (10, 11) are configured to deliver at least one of the first and second aerosols to the canal (3).
S10. The system according to any of the preceding system embodiments wherein the dosing arrangement (10, 11, 40) comprises a first aerosol generator (10, 11) that is configured to vaporize the first and the second components to a first and a second aerosol.
S11. The system according to any of the preceding system embodiments wherein the dosing arrangement (10, 11, 40) comprises a second aerosol generator (11) that is configured to vaporize the second component to a second aerosol.
S12. The system according to any of the preceding system embodiments wherein the dosing arrangement (10, 11, 40) is configured to keep the amount or aerosol constant or essentially constant during an inhale.
S13. The system according to the preceding system embodiment wherein the dosing arrangement (10, 11, 40) is configured to accordingly increase the amount aerosol based on the first component when the amount of aerosol based on the second component is reduced.
S14. The system according to any of the preceding system embodiments wherein the dosing arrangement (10, 11, 40) comprises a controller (40) that is configured to control at least one of the first and second aerosol generators (10, 11).
S15. The system according to the preceding system embodiment wherein the controller (40) is configured to at least control one of the activation(s) of the first and second aerosol generators (10, 11) and the amount of aerosol generated.
S16. The system according to any of the preceding system embodiments wherein at least one of the first and second the aerosol generators (10, 11) are configured to vaporize or aerosolize at least one of the first and second aerosols by heating or ultrasonics.
S17. The system according to any of the preceding system embodiments wherein the dosing component (10, 11, 40) is configured to dose at least the second component in time intervals of at most 100 ms, preferably at most 50 ms, more preferably at most 35 ms, preferably at most 25 ms, more preferably at most 20 ms, more preferably at most 15 ms, even more preferably at most 10 ms and most preferably at most 7.
S18. The system according to any of the preceding system embodiments wherein the dosing component (10, 11, 40) is configured to dose in time intervals of at least 1 ms, preferably at least 2 ms, preferably at least 3 ms, more preferably at least 4 ms, even more preferably at least 5 ms, even more preferably at least 6 ms.
S19. The system according to any of the preceding system embodiments wherein the dosing component (10, 11, 40) is configured to dose in time intervals of between 1 ms and 15 ms, preferably between 2 ms and 20 ms, more preferably between 3 ms and 15 ms and most preferably between 5 ms and 10 ms.
S20. The system according to any of the preceding system embodiments wherein the dosing component (10, 11, 40) is configured to heat at least one of the first component and the second component to a temperature of more than 100° C., preferably at least 150° C., more preferably between 150° C. and 300° C., even more preferably between 180° C. and 260° C. and most preferably between 200° C. and 240° C.
S21. The system according to any of the preceding system embodiments further comprising a boosting member that is configured to overrule the dosing component (10, 11, 40) by a user when activated to change the amount of the second component, particularly to increase the amount of the second component instantly.
S22. The system according to the preceding system embodiment wherein the boosting member comprises at least one of a knob and any associated software switch, such as one installed in a software application installed on an associated handheld device.
S23. The system according to any of the preceding system embodiments wherein the dosing component (10, 11, 40) comprises a reduction model that is configured to cooperate with the controller (40) in the controlling of the delivery of at least one of the first and second components.
S24. The system according to any of the preceding system embodiments wherein the first and second containers (30, 31) and respective first and second aerosol generators (10, 11) are arranged in series along and in connection with a canal (3) for delivering air.
S25. The system according to any of the preceding system embodiments further comprising at least one of a mouthpiece (1) that is configured to be taken into the mouth of a user and a battery (50) that is configured to deliver energy to at least one of the dosing arrangements (10, 11, 40).
S26. The system according to any of the preceding system embodiments wherein the dosing component (10, 11, 40) further comprises a data storage.
S27. The system according to any of the preceding system embodiments further comprising a user interface with at least one of an activation switch, an LED, a display, a fingerprint sensor, a face recognition sensor, a lip recognition sensor.

Below is a list of assembly embodiments. Those will be indicated with a letter “A”. Whenever such embodiments are referred to, this will be done by referring to “A” embodiments.
A1. An assembly comprising the system (100) according to any of the preceding dosing system embodiments and further comprising at least one of a handheld device (200) and a remote server (300) wherein the system is configured to communicate with at least one of the handheld device (200) and the remote server (300).
A2. The assembly according to the preceding embodiment, wherein the wherein the system is configured to communicate data to at least one of the handheld device (200) and the remote server (300).
A3. The assembly according to the preceding embodiment, wherein the system is configured to communicate behavior data to at least one of the handheld device (200) and the remote server (300).
A4. The assembly according to any of the two preceding embodiments, wherein the system is configured to communicate puff data to at least one of the handheld device (200) and the remote server (300).
A5. The assembly according to any of the three preceding embodiments, wherein the system is configured to communicate device settings data to at least one of the handheld device (200) and the remote server (300).
A6. The assembly according to any of the four preceding embodiments, wherein the system is configured to communicate device information data to at least one of the handheld device (200) and the remote server (300).
A7. The assembly according to any of the preceding assembly embodiments, wherein the remote server (300) is configured to communicate data to at least one of the handheld device (200) and the system.
A8. The assembly according to the preceding embodiment, wherein the remote server (300) is configured to communicate reduction algorithm data to at least one of the handheld device (200) and the system.
A9. The assembly according to any of the two preceding embodiments, wherein the remote server (300) is configured to communicate treatment data to at least one of the handheld device (200) and the system.
A10. The assembly according to any of the three preceding embodiments, wherein the remote server (300) is configured to communicate update data to at least one of the handheld device (200) and the system.
A11. The assembly according to any of the preceding assembly embodiments, wherein the handheld device (200) is configured to communicate data to the remote server (300).
A12. The assembly according to the preceding assembly embodiment, wherein the handheld device (200) is configured to communicate behavior data to the remote server (300).
A13. The assembly according to any of the two preceding assembly embodiments, wherein the handheld device (200) is configured to communicate puff data to the remote server (300).
A14. The assembly according to any of the three preceding assembly embodiments, wherein the handheld device (200) is configured to communicate device settings data to the remote server (300).
A15. The assembly according to any of the four preceding assembly embodiments, wherein the handheld device (200) is configured to communicate device information data to the remote server (300).
A16. The assembly according to any of the five preceding assembly embodiments, wherein the handheld device (200) is configured to communicate user data to the remote server (300).
A17. The assembly according to any of the six preceding assembly embodiments, wherein the handheld device (200) is configured to communicate treatment data to the remote server (300).
A18. The assembly according to any of the preceding assembly embodiments, wherein the handheld device (200) is configured to communicate data to the system.
A19. The assembly according to the preceding assembly embodiment, wherein the handheld device (200) is configured to communicate at least one of user identification data and user verification data to the system.
A20. The assembly according to any of the preceding assembly embodiments, wherein the handheld device (200) is configured to communicate user input data to the system.
A21. The assembly according to any of the preceding assembly embodiments, wherein the handheld device (200) is configured to communicate time data to the system.
A22. The assembly according to any of the preceding assembly embodiments, wherein the handheld device (200) is a smart handheld device, such as a smart phone or a tablet computer.
A23. The assembly of the preceding assembly embodiment wherein the dosing system (100) is configured to communicate with the remote server (300) at least one of directly or via the handheld device (200).
A24. The assembly according to any of the preceding assembly embodiments further comprising a software application that is installed on the handheld device (200) that is configured to introduce personalized data of an individual user, such as at least one of age, gender, weight, location, working profiles, smoking habits, number of boost activations per time, and that is making this personalized data available for the training of the reduction model.
A25. The assembly according to the preceding assembly embodiment, wherein the software application is configured to make data communicated to the system available for the training of the reduction model.
A26. The assembly according to any of the two preceding assembly embodiments, wherein the software application is configured to make empirical data available for the training of the reduction model.
A27. The assembly according to any of the preceding assembly embodiments wherein at least one of the dosing system (100) and the handheld device (200) is/are configured to collect user behavior data and to communicate the data to the remote server (300).
A28. The assembly according to the preceding assembly embodiment wherein at least one of the personalized data and the user behavior data is taken as a basis for modifying the reduction model for all users.
A29. The assembly according to the preceding assembly embodiment wherein at least one of the personalized data and the user behavior data is taken as a basis for modifying the reduction model for an individual user.
A30. The assembly according to the preceding assembly embodiment wherein the remote server (300) is configured to collect at least one of user behavior data and personalized data, to compute or modify at least one reduction model and to transfer the reduction model to the dosing system (100), particularly to the dosing component (10, 11, 40).
A31. The assembly according to any of the preceding assembly embodiments, wherein the server is configured to communicate at least one reduction model to the system and wherein the system comprises a software application configured to control the system according to the reduction model.
A32. The assembly according to any of the four preceding assembly embodiments, wherein the server is configured to communicate updates for at least one of the at least one reduction model to the system.
A33. The assembly according to any of the five preceding assembly embodiments, wherein the server is configured to adapt at least one of the at least one reduction model based on data received from at least one of the system, the handheld device, a plurality of the systems and a plurality of the handheld devices, each corresponding to at least one system.
A34. The assembly according to the preceding assembly embodiment wherein the handheld device (200) is configured to display data of a plurality of users.
A35. The assembly according to any of the preceding assembly embodiments wherein the system according to any of the preceding system embodiments is configured to be controlled by a remote controller (400).
A36. The assembly according to the preceding assembly embodiment wherein the remote controller (400) is configured to connect directly with the system or indirectly with the system over at least one of the handheld device (200) and a remote server (300) and the cloud (300).
A37. The assembly according to the preceding assembly embodiment wherein the remote controller is configured to control the dosing arrangement (10, 11, 40), particularly the dosage of the second component to be delivered.
A38. The assembly according to any of the preceding two assembly embodiments wherein the remote controller (400) is configured to control by at least one encrypted command and at least one of the system and the handheld device (200) are configured to decrypt the command and to provide the command to the controller of the system for controlling the dosing arrangement (10, 11, 40).
A39. The assembly according to any of the three preceding assembly embodiments wherein the remote controller (400) is at least one of a computer, a tablet and a second handheld device.
A40. The assembly according to any of the five preceding assembly embodiments, wherein the remote controller (400) is the handheld device (200).
A41. The assembly according to the preceding assembly embodiment wherein the remote controller (400) and the system are configured to communicate with each other hardwired or wireless.
A42. The assembly according to the preceding assembly embodiment wherein the remote controller (400) and the system are configured to communicate at least over one of wifi, Bluetooth, nfc.

Below is a list of method embodiments. Those will be indicated with a letter “M”. Whenever such embodiments are referred to, this will be done by referring to “M” embodiments.
M1. A method for dosing a composition of an inhaling substance in an inhaler, the composition consisting of at least two components, the method comprising the steps of:
   a. providing a first component of the composition of the inhaling substance during inhaling;
   b. providing a second component of the composition of the inhaling substance during inhaling;
   c. wherein at least the second component is dosed in intervals of at most 50 ms during inhaling.
M2. A method for dosing a composition of an inhaling substance in an inhaler, the composition consisting of at least two components, the method comprising the steps of:
   a. providing a first component of the composition of the inhaling substance;
   b. providing a second component of the composition of the inhaling substance;
   c. detecting a number of inhales;
   d. wherein at least the second component is automatically reduced over time.
M3. Method combining the two preceding method embodiments.
M4. Method according to any of the above two method embodiments wherein the second component is automatically reduced over time by reducing the number of inhales comprising the second component.
M5. Method according to any of the above three method embodiments wherein the second component is reduced by delivering the second component in time intervals during one inhale and by reducing the number of time intervals for a single inhale over time.
M6. Method according to any of the preceding method embodiments wherein the inhaler is a mobile device, preferably a battery driven mobile device.
M7. Method according to any of the preceding method embodiments wherein the inhaler is an electronic cigarette.
M8. The method according to any of the preceding method embodiments wherein at least the first component is a liquid to be vaporized to provide an aerosol as a carrier component together with air.
M9. The method according to any of the preceding method embodiments further comprising delivering air from a canal to a mouthpiece wherein at least one of the first and second aerosols are delivered to the canal.
M10. The method according to the preceding method embodiment wherein the first and the second aerosols are delivered in series to the canal.
M11. The method according to the preceding method embodiment wherein at least one of the first and second aerosols are directly delivered into the canal.
M12. The method according to any of the preceding method embodiments wherein at least the first liquid and/or the second liquid are vaporized.
M13. The method according to any of the preceding method embodiments wherein at least the first liquid and/or the second liquid are vaporized by at least one of heating and ultrasonic.
M14. The method according to the preceding method embodiment wherein the heating is done by at least a porous component releasing at least one of the first and the second component as an aerosol when being heated.
M15. The method according to the preceding method embodiment wherein the porous component comprises a rare earth material with a porosity gradient that emits vapor when being heated.
M16. The method according to any of the preceding method embodiments wherein the heating is done by at least a fibrous component releasing at least one of the first and the second component as an aerosol when being heated.
M17. The method according to any of the preceding method embodiments wherein the time interval is at most 100 ms, preferably at most 50 ms, more preferably at most 35 ms, preferably at most 25 ms, more preferably at most 20 ms, more preferably at most 15 ms, even more preferably at most 10 ms and most preferably at most 7.
M18. The method according to any of the preceding method embodiments wherein the time interval is at least 1 ms, preferably at least 2 ms, preferably at least 3 ms, more preferably at least 4 ms, even more preferably at least 5 ms, even more preferably at least 6 ms.
M19. The method according to the previous method embodiment wherein the time interval is between 1 ms and 15 ms, preferably between 2 ms and 20 ms, more preferably between 3 ms and 15 ms and most preferably between 5 ms and 10 ms.
M20. The method according to any of the preceding method embodiments wherein at least one of the first component and the second component is/are heated to a temperature of more than 100° C., preferably at least 150° C., more preferably between 150° C. and 300° C., even more preferably between 180° C. and 260° C. and most preferably between 200° C. and 240° C.
M21. The method according to any of the preceding method embodiments wherein an overruling controlling is configured for activation by a user to change the amount of the second component, particularly to increase the amount of the second component instantly.
M22. The method according to the preceding method embodiment wherein the overruling controlling can be activated by a knob configured at the inhaler and/or any associated software switch, such as one installed in an application smart handheld.
M23. The method according to any of the preceding method embodiments wherein the second component is being controlled by a reduction model wherein a control is controlling the delivery of the second component by the reduction model.
M24. The method according to any of the preceding method embodiments wherein user behavior data is collected and taken as a basis for modifying the reduction model.
M25. The method according to the preceding method embodiment wherein the user behavior data, such as number of overruling controlling activations by the user, is taken as a basis for modifying the reduction model for all users.
M26. The method according to any of the preceding method embodiments wherein the user behavior data, such as number of overruling controlling activations by a plurality of users and the individual user, is taken as a basis for modifying the reduction model for the individual user
M27. The method according to the preceding method embodiment wherein the inhaler is associated with a software application that is installed on a smart handheld device that is configured to introduce personalized data of an individual user, such as at least one of age, gender, weight, location, working profiles, smoking habits and that is making this personalized data available for the training of the reduction model.
M28. The method according to the preceding embodiment wherein data is collected from a plurality of uses or users and computed remotely, such as in the cloud.
M29. The method according to the preceding embodiment wherein the data of a plurality of users or uses is graphically compared and displayed to user in the software application.
M30. The method according to any of the preceding method embodiments with the step of controlling the system according to any of the preceding system embodiments by a remote controller.
M31. The method according to the preceding method embodiment wherein the remote controller is controlling the dosing component, particularly the dosage of the second component.
M32. The method according to the preceding method embodiment wherein the remote controller connects directly with the system or indirectly with the system over at least one of the handheld device and a remote server and the cloud.
M33. The method according to any of the preceding method embodiments wherein the remote controller controls by at least one encrypted command and at least one of the system and the handheld device that decrypt the command and provide the command to the controller of the system for controlling the dosing arrangement, particularly the dosage of the second component.
M34. Therapeutic method with the step of applying any of the preceding system embodiments or method embodiments.

Below is a list of use embodiments. Those will be indicated with a letter “U”. Whenever such embodiments are referred to, this will be done by referring to “U” embodiments.
U1. Use of any of the preceding system embodiments or method embodiments for the treatment of nicotine or cannabis addiction.
U2. Use of any of the preceding system embodiments or method embodiments for the treatment of a pain-related disease.
U3. any of the preceding system embodiments or method embodiments for the treatment of any of multiple sclerosis, neuropathic disease, rheumatic disease, anorexia, AIDS, cancer, chemotherapy, Tourette syndrome, psychosis and/or Alzheimer disease.

### BRIEF DESCRIPTION OF THE DRAWINGS

Further potential and thus non-limiting features, details and advantages of the invention will be discussed in the drawings are shown clearly.

FIG. 1 shows an example of an e-cigarette known in the art.

FIG. 2 shows another embodiment of an e-cigarette according to the present invention in a schematic manner and in part only.

FIG. 3 exemplifies another embodiment of an e-cigarette according to the present invention is a schematic manner and in part.

FIG. 4 depicts another embodiment 3 of an e-cigarette according to the present invention in a schematic manner and in part.

FIG. 5 shows another embodiment of an e-cigarette according to the present invention in a schematic manner and in part.

FIG. 6 exemplifies another embodiment of an e-cigarette according to the present invention in a schematic manner and in part.

FIG. 7 shows another embodiment of an e-cigarette according to the present invention schematically and in part.

FIG. 8 illustrates an embodiment of a mobile device functionally arranged to the e-cigarette according to any of the embodiments of the preceding figures and preferably a remote server and further preferably a remote controller.

FIG. 9 demonstrates an embodiment of a reduction model underlying an e-cigarette according to the present invention.

FIG. 10 exemplifies an embodiment of a cartridge for an e-cigarette according to the present invention.

FIG. 11 shows a flow path representing an embodiment for the control of an e-cigarette according to the present invention.

### DETAILED DESCRIPTION

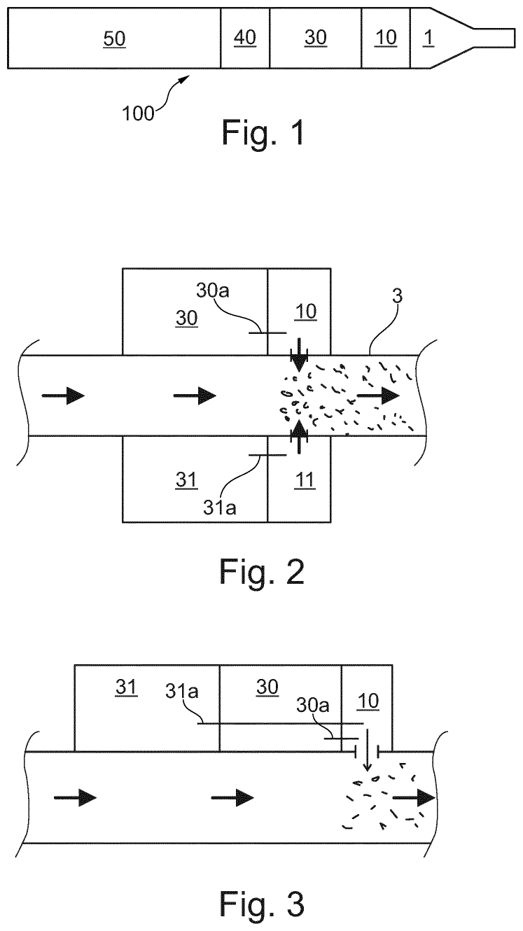

FIG. 1 shows an example of an e-cigarette 100 as common in the art. This is just shown for illustrative purposes. A mouthpiece 1 can have any shape and can be made of a number of materials that can be used for human beings. In the embodiment shown it is of generally conical shape. However, as the embodiment is a sketch only, the shape can vary to be able to better adapt to the needs of a user or to provide a more esthetic appearance.

An aerosol generator 10 can be placed upstream to the mouthpiece 1. In the embodiment shown it is place directly in the neighborhood of the mouthpiece but It can also be placed further upstream of the flow of vapor or aerosol.

A container or tank 30 is usually arranged closed to the aerosol generator as a liquid forming a component to be vaporized by the aerosol generator 10 can be delivered to the aerosol generator 10 more easily. However, the container 30 can be arranged somewhere else as well.

A controller 40 can also be integrated into the e-cigarette. The controller usually controls the power supply and the component delivered to the aerosol generator 10. It usually comprises a CPU, a storage, an optional interface etc. and can be an integrated component.

An energy storage or battery 50 provides the power supply and can have any shape, can be re-chargeable, can also have an external power supply or connection.

FIG. 2 shows an example of a part or portion of an e-cigarette according to the present invention. This figure is schematic as it functionally shows a potential embodiment that can be arranged or designed differently. At least two containers 30, 31 can be provided and each can contain a component for the composition to be inhaled. They deliver the respective components by container connectors 30a, 31a, respectively.

A canal 3 for guiding air and/or vapor is shown with the general flow of gas and/or vapor shown with arrows.

A first aerosol generator 10 can be connected or assembled to the first container 30 in order to vaporize the content in the first container 30. A second aerosol generator 11 can be connected or assembled to the second container 31 in order to vaporize the content in the second container 31. The vapor generated in the first aerosol generator 10 and/or the vapor in the second aerosol generator 11 can be fed into the gas and/or vapor stream of the e-cigarette either together or independent of each other. This is schematically shown by the little arrows and the resulting aerosol drops leaving the aerosol generators 10, 11.

Both containers 30, 31 can be chargeable with new components or can be replaced by other containers (not shown) containing the same or different liquids. The containers can be housed at least in part by a housing (not shown) or can be attached from the exterior to or adjacent to the canal 3. They can be snap-fitted, locked, screwed or attached in any manner. The containers 30, 31 can be reusable or disposable. They can have many and even different shapes as will be exemplified later. They can be transparent as well in order to allow the user to see the amount of remaining liquid. The liquid(s) can be colored in order to assist the inspection of the level. In case there is a housing around the container(s) one or more windows within the housing can be provided to allow a view onto the containers. This can also serve the inspection to see whether or not the e-cigarette is properly filled with containers. Color codes of the containers can also serve to immediately make it apparent what they contain.

The aerosol generators 10, 11 can work according to the same or according to different principles and/or can be configured to deliver different amounts of vapor according to the components, their consistency and/or the amount to be fed into the gas stream. The latter can be particularly controlled by a number of shots or releases or intervals during one inhaling cycle of a user. The numbers of intervals can be controlled and can be pre-defined or individually defined by software and/or hardware components of the controller 40.

The embodiment shown in FIG. 3 exemplifies another embodiment according to which the containers 30, 31 can be arranged sequentially. In the embodiment shown they are arranged one behind the other. They can also be arranged in parallel to each other or on different sides of the aerosol generator 10 depicted in this embodiment. All those embodiments have in common that the containers 30, 31 deliver their components to one aerosol generator 10. In the embodiments shown each container connector 30a, 30b delivers the respective first and second components to the one aerosol generator 10. This aerosol generator can then be configured to generate aerosol using each component individually or together.

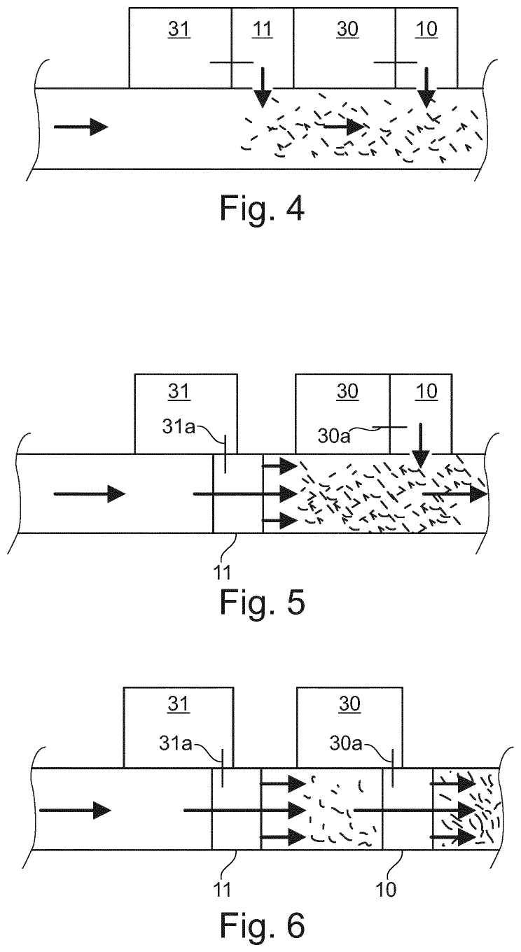

FIG. 4 depicts an embodiment with a sequential arrangement of the first aerosol generator 10 with the respective first container 30 and stream upwards the second aerosol generator 11 with the respective second container 31. One or both container(s) 30, 31 can be arranged also on different sides of the respective aerosol generator 10, 11, respectively or even on different sides with respect to the canal 3. As will be addressed later, the aerosol generators can also be arranged at least in part in the canal 3.

A similar arrangement of components is shown in FIG. 5. As an example the first aerosol generator 10 is arranged outside the canal 3 while the second aerosol generator 11 is assembled at the side or circumferentially around canal 3. The reason for this can be different consistencies or amounts to be delivered of the components contained in the different containers 30, 31. It is also apparent that the amount of aerosol will change within the canal with the direction of flow which may justify the different arrangements of aerosol generators 10, 11 first within the canal and second at the side of the canal 3.

According to FIG. 6, both aerosol generators 30, 31 are connected to aerosol generators 10, 11 that are arranged within, in part within and/or substantially within canal 3.

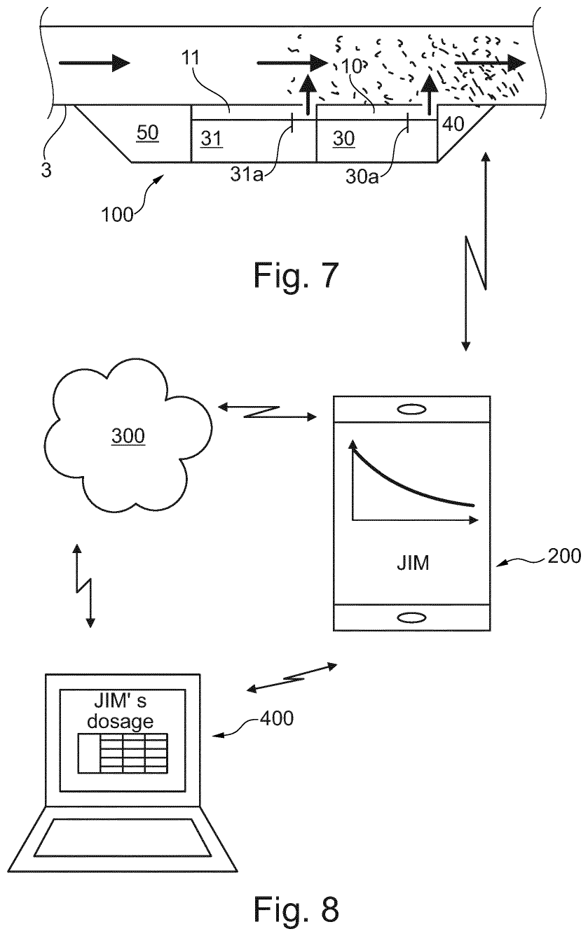

Still another embodiment is shown in FIG. 7. According to the embodiment shown the controller 40 and energy source 50 are more integrally arranged with other components at or around the canal 3. They can also be arranged together or oppositely to the embodiment shown.

The containers 30, 31 are arranged to the outside of the e-cigarette or are flush with the contours thereof or a housing. In this case they can be more easily replaced.

The aerosol generators 10, 11 are arranged radially more inwardly to the containers, or at least in part thereof. The arrangement may further deviate with the circumference of the e-cigarette. The aerosol generators 10, 11 can be further arranged within the canal 3. The size of canal 3 compared with the other components can also be smaller or substantially smaller.

This embodiment can then be connected to a smart handheld device, such as a smart phone or smart tablet. A software application can be installed on the handheld device in order to communicated either unidirectionally from the e-cigarette to the handheld device or vice versa or bidirectionally. The latter allows a more active and interesting exchange of data and user input. As mentioned before, the software application can also comprise a switching function activating a “boosting function” that allows a user to instantly inhale a larger or large amount of the second component, such as nicotine. This can happen in case the user is in a situation where he or she needs or enjoys more nicotine. This could be at a party or under certain stress or mental workload.

When the activation of the boosting functions happens the software application could also track that and can compute this information locally or can feed this information to a remote device, such as a server, in order to store and compute it. One aspect is shown on the left hand of FIG. 8 where a communication of the handheld 200 with the cloud 300 (remote computing or remote server) is shown. Also, the e-cigarette 100 could communicate directly with the cloud 300. In any case other information from other users can also be used and computed. In case of a plurality of users or even a large group of users, certain patterns can be determined and the reduction model can be adapted accordingly. The adaption could be that at weekends at evenings more nicotine is delivered than usually or—in other words—the nicotine level can be lowered preferably over working days.

The information can also be associated with other information, such as gender, age, weight, location, work profile, smoking habits, etc.

A social media platform can also be affiliated or erected where users compare their progress of minimizing the consumption of the second component, such as nicotine. The presence of more information as mentioned before could then also automatically group or affiliate users and can even suggest an exchange of data or comparison so that users are further motivated to reduce the consumption of the second component. As an example, users in a certain region or of similar profiles can be compared or can compare them upon agreement.

The system can be controlled by a remote controller 400 exclusively or additionally. This is shown as an option in the lower part of FIG. 8.

The remote controller 400 can be configured to connect directly with the system 100 or indirectly with the system 100 over at least one of the handheld device 200 and a remote server 300 and the cloud 300. The latter has the advantage that for example a doctor who is in possession of the remote controller can monitor or follow the use of the system 100 by a user and can even adapt the consumption of the second component accordingly.

The remote controller is shown to be a computer but can be any other suitable device, such as a tablet or second handheld device.

The configuration can be hardwired or wirelessly. In case it is hardwired a special and rather uncommon interface can be provided in order to limit accessibility. Alternatively or additionally an encryption can take place. However, in the embodiment shown a wireless and bidirectional data transmission is shown as an example only.

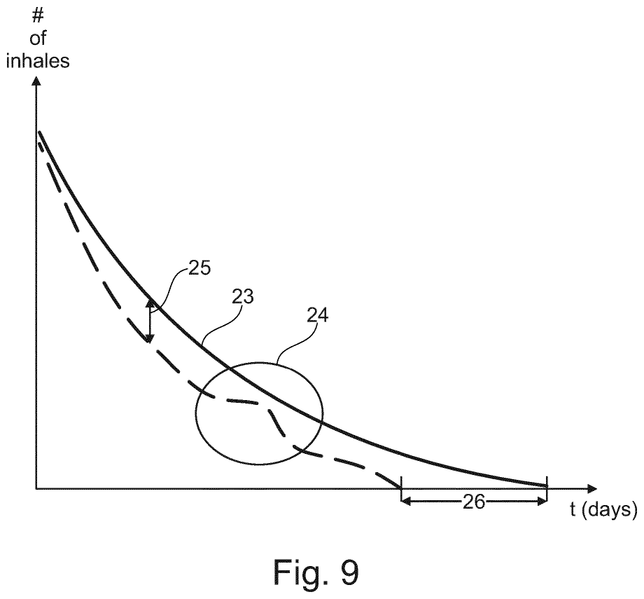

FIG. 9 shows a potential correlation of the number of inhales with a certain component, such the second component or nicotine, over time, such as days. As in one example the number of inhales with the second component is reduced, the amount goes first more quickly and then more gradually to zero or close to zero. Alternatively or additionally, the ordinate could also determine the amount of the second component or nicotine over the time. As mentioned before the amount of the second component can also be reduced by pulsing the donation of the second component during one or more inhales and then reduce the numbers of pulses during one or multiple inhalations with the second component.

Anyhow, FIG. 9 shows the pre-determined or ideal decrease curve 23 of the second component over time. Alternatively or additionally, the actual decrease curve 24 can also be tracked. Also any deviation 25 in consumption of the second component or any gain (or loss) in time 26 can be communicated or visualized. This can be displayed on the handheld device to the user in order to motivate him or her. In the example shown the user would be faster than pre-determined and he or she could be displayed or communicated appreciation. In case the user allows, this appreciation could also be communicated to other users on a software platform as described.

In the example shown the individual consumption of the second component has substantially deviated in section 24. This can also be particularly highlighted to the user, either retrospectively and/or prognostically. The latter can start to inform the user when the consumption starts to considerably change from the forecast.

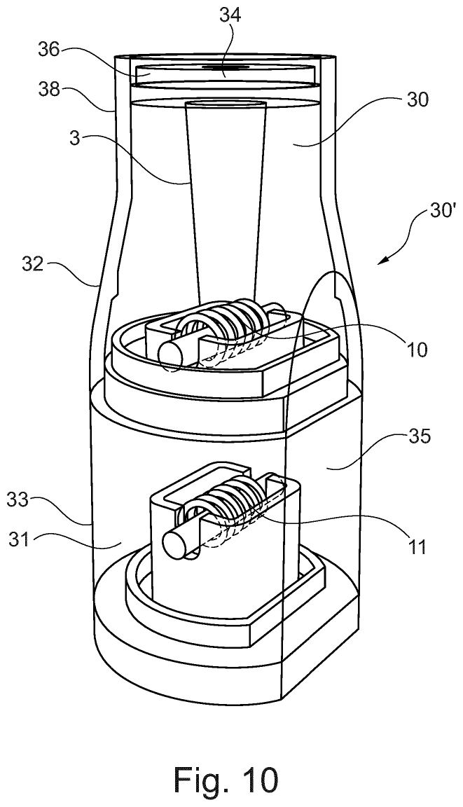

FIG. 10 shows a cartridge 30′ containing the first or second component. The cartridge 30′ can at the same time comprise the containers 30, 31. The cartridge 30′ can comprise any shape, it preferably has a shape so that the cartridges cannot be mixed with other available cartridges 30′ and that the position of the cartridge in a system as specified is determined. The shape can be unsymmetrical and can comprise a flat side surface 35. This is an example only. The cartridge 30′ can also comprise a bottom 34, a cylindrical part 33 (except from the flat side 35). The can be also a truncated cone part 32 connecting the base part with a neck portion 38. The neck portion 38 can be of generally cylindrical shape in order to allow a easier insertion of the cartridge 30′ into the system, the assembled e-cigarette or body thereof. This can be similar to an ink cartridge for a fountain pen.

An open end can then comprise a valve and sealing structure 36 with an opening that allows the delivery of the component contained in the cartridge 30′ to the e-cigarette in the assembled state. The valve and sealing structure can comprise a tamper evident structure, as well, in order to allow the indication whether or not the cartridge has been used before. Additionally or alternatively, the cartridge 30′ can be configured to be re-fillable.

As can also be seen, the air canal 3 is arranged centrally to that in the example shown the air would flow or be sucked from the bottom to the top through the cartridge 30′. Upstream the container 31 is arranged that delivers the second component to the air flow in canal 3. The second component in the second container 31 is aerosolized by the second aerosol generator 11 that is at least in part arranged in the air canal 31. In the embodiment shown a coil spring is heating the second component that is sucked by a capillary effect to or into the coil. As said, any other principle or arrangement can be used as well.

The first container 30 is arranged on top or downstream of the second container 31. It can also be the other way. The first container is then delivering the first component into the air stream in canal 3 as an aerosol. The first aerosol generator 10 can thus be arranged, similar to the second one, at least in part in the air canal. Preferably, the first component is delivered in use by the user in order to have aerosol in the air or wet air. In case of a dry puff, this would be immediately realized by the user and would be provide discomfort, and the user would know that no component is delivered, particularly not the second component that he may be addicted to.

The system could be also controlled to have either the first component or the second component contained in the air flow. However, in case the first component would comprise a tastable flavor and the second a component that cannot be tasted but is therapeutically active the user would also realize.

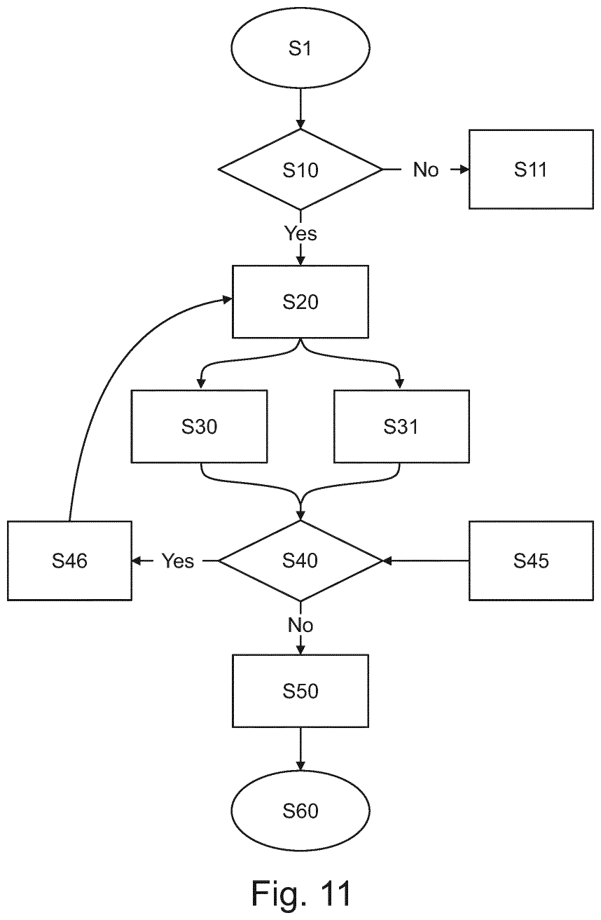

FIG. 11 exemplifies a flow chart of a control of the e-cigarette according to the invention, at least in part. A control start S1 can be activated by a user and/or any kind of sensor can automatically turn the power on whenever it senses potential use of a user. The sensor can be an accelerometer etc. According to step S10 the device can check the availability of sufficient power, e.g. to energize the aerosol generator. In the negative, the user will be noted in step S11. This can take place by an LED, and audio signal, a display, a display of a remote device etc.

In case of sufficient power the device can be started in step S20. In an embodiment one of the reduction model, the present status of the user, the date, the time, the content of one or more cartridges or containers can be determined regarding the amount, the kind of component etc. In a more simple device just the aerosol generators can be heated up and the components are delivered to the aerosol generators by respective valve control. Anything dosing the components to the aerosol generators can be called valve in this context.

In an optional step S40 the input of any particular signal can be detected, such as in step S45 the pressing of a knob of the user, in a step 46 the model for the delivery of the components can be re-determined. This can be done by an optional element, such as a physical knob or a software control in the e-cigarette or smart device associated with the e-cigarette. This can be an overruling command, e.g. to increase the amount of one component more, such as the nicotine. This can be user friendly in case the user feels it necessary to increase the level of nicotine for any given reasons, such as when attending a party. However, this is optional only and can involve further or alternative measures as well.

In case no such interference happens, the e-cigarette continues its operation by the dosing of the components according to the reduction model until it is not used or switched off in step S60.

It is to be noted that other embodiments with further different arrangements of structural components are covered by the present invention.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of a first option and a second option” is intended to mean the first option or the second option or the first option and the second option.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be the preferred order, but it may not be mandatory to carry out the steps in the recited order. That is, unless otherwise specified or unless clear to the skilled person, the orders in which steps are recited may not be mandatory. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

1-18. (canceled)

19. A system for dosing an inhaling composition, the system comprising:
a. a first container for a first component of the composition;
b. a second container for a second component of the composition; and
c. a dosing arrangement that is configured to dose at least the second component in time intervals of at most 200 ms;
wherein
d. the dosing arrangement is configured to automatically reduce the dose of at least the second component over time; and
e. the system is a mobile device, preferably a battery driven mobile device and more preferably an electronic cigarette or e-cigarette.

20. The system according to claim 19 wherein the dosing arrangement is configured to reduce a number of inhales comprising the second component, preferably by a reduction model, and/or
wherein the dosing arrangement is configured to reduce at least one of an amount of the second component over a number of inhales and a number of doses of the second component during an inhale.

21. The system according to claim 19, wherein
a. the dosing arrangement comprises
i. a first aerosol generator that is configured to vaporize the first component to a first aerosol, preferably so as to provide a first aerosol as a carrier component together with air; and
ii. a second aerosol generator that is configured to vaporize the second component to a second aerosol; and/or
iii. a data storage; and wherein
b. the system comprises a controller that is configured to control at least one of the first and second aerosol generators.

22. The system according to claim 19, further comprising a boosting member that is configured to overrule the dosing component by a user when activated to change the amount of the second component, particularly to increase the amount of the second component instantly.

23. The system according claim 19, wherein the dosing component is configured to dose at least the second component in time intervals of at most 100 ms, preferably at most 50 ms, more preferably at most 35 ms, preferably at most 25 ms, more preferably at most 20 ms, more preferably at most 15 ms, even more preferably at most 10 ms and most preferably at most 7, and/or in time intervals of at least 1 ms, preferably at least 2 ms, preferably at least 3 ms, more preferably at least 4 ms, even more preferably at least 5 ms, even more preferably at least 6 ms.

24. An assembly comprising the system according to claim 19 and further comprising at least one of a handheld device and a remote server wherein the system is configured to communicate with at least one of the handheld device and the remote server.

25. The assembly according to claim 24 further comprising a software application that is installed on the handheld device that is configured to introduce personalized data of an individual user, such as at least one of age, gender, weight, location, working profiles, smoking habits, number of boost activations per time, and that is making this personalized data available for the training of the reduction model.

26. The assembly according to claim 25 wherein at least one of the personalized data and the user behavior data is taken as a basis for modifying the reduction model for all users.

27. The assembly according claim 26 wherein at least one of the personalized data and the user behavior data is taken as a basis for modifying the reduction model for an individual user.

28. The assembly according to claim 27 wherein the system is configured to be controlled by a remote controller that is preferably configured to control the dosing arrangement, particularly the dosage of the second component to be delivered.

29. The assembly according to claim 27 wherein the remote controller is configured to control by at least one encrypted command and at least one of the system and the handheld device are configured to decrypt the command and to provide the command to the controller of the system for controlling the dosing arrangement.

30. A method for dosing a composition of an inhaling substance in an inhaler, the composition consisting of at least two components, the method comprising the steps of:
a. providing a first component of the composition of the inhaling substance during inhaling;
b. providing a second component of the composition of the inhaling substance during inhaling;
c. wherein at least the second component is dosed in intervals of at most 50 ms during inhaling.

31. A method for dosing a composition of an inhaling substance in an inhaler, the composition consisting of at least two components, the method comprising the steps of:
a. providing a first component of the composition of the inhaling substance;
b. providing a second component of the composition of the inhaling substance;
c. detecting a number of inhales;
d. wherein at least the second component is automatically reduced over time.

32. A method comprising
a. providing a first component of the composition of the inhaling substance;
b. providing a second component of the composition of the inhaling substance;
c. detecting a number of inhales;
d. wherein at least the second component is automatically reduced over time;
wherein an overruling controlling is configured for activation by a user to change the amount of the second component, particularly to increase the amount of the second component instantly, and wherein at least the second component is dosed in intervals of at most 50 ms during inhaling.

33. The method according to claim 30, wherein
a. the second component is being controlled by a reduction model wherein a control is controlling the delivery of the second component by the reduction model;
b. user behavior data is collected and taken as a basis for modifying the reduction model;
c. the inhaler is associated with a software application that is installed on a smart handheld device that is configured to introduce personalized data of an individual user, such as at least one of age, gender, weight, location, working profiles, smoking habits and that is making this personalized data available for the training of the reduction model; and
d. data is collected from a plurality of uses or users and computed remotely.
